(19) United States
(12) Patent Application Publication
(10) Pub. No.: US 2015/0322452 A1
(43) Pub. Date: Nov. 12, 2015

(54) COMPOSITIONS AND METHODS FOR INCREASING PLANT GROWTH AND YIELD

(71) Applicant: Donald Danforth Plant Science Center, St. Louis, MO (US)

(72) Inventors: Lin Wang, St. Louis, MO (US); Thomas P. Brutnell, St. Louis, MO (US); Todd C. Mockler, St. Louis, MO (US); Douglas W. Bryant, St. Louis, MO (US)

(73) Assignee: DONALD DANFORTH PLANT SCIENCE CENTER, St. Louis, MO (US)

(21) Appl. No.: 14/708,823

(22) Filed: May 11, 2015

Related U.S. Application Data
(60) Provisional application No. 61/991,949, filed on May 12, 2014.
(60) Provisional application No. 62/023,432, filed on Jul. 11, 2014.

(51) Int. Cl. C12N 15/82; C07K 14/415

(52) U.S. Cl. CPC C12N 15/8261; C12N 15/8222; C07K 14/415; C12N 2830/002

(57) ABSTRACT

Compositions and methods for increasing plant yield are disclosed. Compositions comprise transcription factors that find use in modulating the expression of a gene or nucleotide sequence of interest in a plant. Additionally, promoters and cis-regulatory elements are disclosed that may be used to drive the expression of a nucleotide sequence of interest in a plant. Methods for the use of such compositions as well as transformed plants are disclosed.

### CROSS-REFERENCE TO RELATED APPLICATIONS

This application claims the benefit of U.S. Provisional Application No. 61/991,949, filed May 12, 2014, and U.S. Provisional Application No. 62/023,432, filed Jul. 11, 2014, the contents of both applications are herein incorporated by reference in their entirety.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 462647SEQLIST.txt, created on May 7, 2015, and having a size of 1,274 KB and is filed concurrently with the specification. The sequence listing contained in this ASCII formatted document is part of the specification and is herein incorporated by reference in its entirety.

### FIELD OF THE INVENTION

The invention is drawn to compositions and methods for controlling gene expression involved in plant growth and development.

### BACKGROUND OF THE INVENTION

The ever-increasing world population and the dwindling supply of arable land available for agriculture fuels research towards developing plants with increased biomass and yield. Conventional means for crop and horticultural improvements utilize selective breeding techniques to identify plants having desirable characteristics. However, such selective breeding techniques have several drawbacks, namely that these techniques are typically labor intensive and result in plants that often contain heterogeneous genetic components that may not always result in the desirable trait being passed on from parent plants. Advances in molecular biology provide means to modify the germplasm of plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

Traits of interest include plant biomass and yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigor may also be important factors in determining yield. Optimizing the abovementioned factors may therefore contribute to increasing crop yield.

An increase in seed yield is a particularly important trait since the seeds of many plants are important for human and animal consumption. Crops such as corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain. An increase in plant biomass is important for forage crops like alfalfa, silage corn and hay. Many genes are involved in plant growth and development. Therefore, methods are needed for modulating such genes.

### SUMMARY OF THE INVENTION

Compositions and methods for increasing plant growth for higher crop yield are provided. Compositions comprise transcription factors and enhancers. The transcription factors can be used to alter plant growth by modulating the expression level and/or expression pattern of one or more genes of interest in a plant. Transcription factors that regulate genes involved in plant growth can be modulated to increase plant growth, increase plant mass, and plant yield. Enhancer elements or cis-regulatory elements are provided that may be used to alter the expression of a downstream open reading frame, whether said open reading frame encodes a transcription factor or any other gene of interest. Such enhancer elements and transcription factors can be used alone or in combination.

The invention further comprises synthetic promoters and promoter elements. Such promoters are useful for expressing nucleotide sequences of interest. DNA constructs comprising the elements of the invention, plants, and plant parts transformed with such constructs are provided.

Embodiments of the invention include:

1. A method of improving plant growth by altering the expression of at least one nucleotide sequence encoding a transcription factor (TF) listed in Table 1, or a fragment or variant thereof.
2. The method of embodiment 1 wherein the at least one transcription factor is upregulated such that expression of the TF is increased relative to a control plant cell.
3. The method of embodiment 1 wherein the at least one transcription factor is downregulated such that expression of the TF is decreased relative to a control plant cell.
4. The method of embodiment 1, 2, or 3 wherein said altering is achieved by the stable insertion of at least one expression construct comprising a promoter that drives expression in a plant cell, operably linked to at least one nucleotide sequence encoding at least one transcription factor of Table 1, or a fragment or variant thereof.
5. The method of any one of embodiments 1-4, wherein said fragment or variant has at least 80% sequence identity to the TF, wherein said nucleotide sequence retains transcription factor activity.
6. The method of any one of embodiments 1-4, wherein said fragment or variant has at least 90% sequence identity to the TF, wherein said nucleotide sequence retains transcription factor activity.
7. The method of any one of embodiments 1-4, wherein said fragment or variant has at least 95% sequence identity to the TF, wherein said nucleotide sequence retains transcription factor activity.
8. The method of any one of embodiments 1-3, wherein said altering is achieved by stable insertion of a DNA construct comprising at least one promoter that drives expression in a plant cell, operably linked to one or more amiRNA cassettes designed to target at least one transcription factor of Table 1.
9. The method of any one of embodiments 1-3 wherein said altering is achieved by stable insertion of a transformation construct comprising at least one promoter that is operable in a plant cell, operably linked to at least one RNAi cassettes designed to target at least one transcription factor of Table 1.
10. The method of any one of embodiments 1-3 wherein said altering is achieved by transforming a plant species of interest with a self-replicating transformation construct derived from a plant virus and comprising at least one promoter that drives expression in a plant cell, operably linked to at least one open reading frame encoding a transcription factor of Table 1.
11. The method of any one of embodiments 1-3 wherein said altering is achieved by transforming a plant species of interest with a self-replicating transformation construct derived from a plant virus and comprising at least one promoter that is operable in a plant cell, operably linked to one or more amiRNA cassettes designed to target a transcription factor of Table 1.
12. The method of any one of embodiments 1-3 wherein said altering is achieved by transforming a plant species of interest with a self-replicating transformation construct derived from a plant virus and comprising at least one promoter that drives expression in a plant cell, operably linked to at least one RNAi cassettes designed to target a transcription factor of Table 1.
13. The method of any one of embodiments 1-3 wherein said altering is achieved by inserting at least one cis-regulatory element into the genome of a plant cell, at a location such that the cis-regulatory elements alters the expression level and/or expression profile of a TF of Table 1, wherein said at least one cis-regulatory element comprises a nucleotide sequence having at least 90% sequence identity to the elements set forth in SEQ ID NOs: 475-536 and 543.
14. The method of embodiment 13, wherein said at least one cis-regulatory element comprises the nucleotide sequence set forth in SEQ ID NOs: 475-536 and 543.
15. The method of any one of embodiments 8-14 wherein the promoter is a constitutive promoter.
16. The method of any one of embodiments 8-14 wherein the promoter is a non-constitutive promoter.
17. The method of embodiment 16 wherein the promoter is a developmentally-regulated promoter.
18. The method of embodiment 16 wherein the promoter is a circadian-regulated or diurnally-regulated promoter.
19. The method of embodiment 16 wherein the promoter is a tissue-specific promoter.
20. The method of embodiment 16 wherein the promoter is an inducible promoter.
21. The method of embodiment 16 wherein the promoter is a light-regulated promoter.
22. A synthetic promoter operable in a plant cell comprising at least one cis-regulatory element selected from the elements set forth in SEQ ID NOs: 475-536 and 543 operably linked to at least one core promoter element that is operable in a plant cell.
23. The synthetic promoter of embodiment 22 that comprises SEQ ID NO: 1 or a sequence with at least 80% homology to SEQ ID NO: 1
24. A method of altering the expression of at least one gene of interest in a plant cell by inserting into the genome of a plant cell a construct comprising the synthetic promoter of embodiment 22 operably linked to said at least one gene.
25. A method of altering the expression of at least one gene of interest in a plant cell comprising inserting into the genome of a plant cell a construct comprising the synthetic promoter of embodiment 22 operably linked to an amiRNA cassette designed to target the at least one gene of interest.
26. A method of altering the expression of one or more genes of interest in a plant cell by inserting into the genome of a plant cell a construct comprising the synthetic promoter of embodiment 22 operably linked to an RNAi cassette designed to target the at least one gene of interest.
27. A method of altering the expression of at least one gene of interest in a plant cell comprising inserting at least one cis-regulatory element set forth in SEQ ID NOs: 475-536 and 543 into a plant genome at a location proximal to said at least one gene to alter the expression of said at least one gene of interest.
28. The method of embodiment 27 wherein at least one cis-regulatory element is inserted upstream of a core promoter element of a gene of interest and increases expression of said gene of interest.
29. The method of embodiment 27 wherein at least one cis-regulatory element is inserted upstream of a core promoter element of a gene of interest and alters the expression profile of said gene of interest.
30. The method of any one of embodiments 1-21 and 26-29, wherein the plant of interest is a monocotyledonous plant.
31. The method of any one of embodiments 1-21 and 26-29, wherein the plant of interest is a dicotyledonous plant.
32. The method of any one of embodiments 22-25 wherein the synthetic promoter comprises the sequence of SEQ ID NO: 1 or a sequence with at least 80% homology to SEQ ID NO: 1.
33. An isolated polynucleotide or a recombinant DNA comprising a nucleotide sequence encoding a polypeptide having at least 90% identity to the amino acid sequences of the TFs set forth in Table 1.
34. The isolated polynucleotide or recombinant DNA of embodiment 33, wherein said polypeptide has at least 95% identity.
35. The method of any one of embodiments 1-3, wherein said altering is achieved by the expression of a gene encoding a dCas9 protein fused to a domain for transcriptional regulation.
36. A synthetic promoter operable in a plant cell comprising at least one of the cis-regulatory elements set forth in SEQ IDs No. 475-536 and 543 operably linked to at least one core promoter element operable in a plant cell.
37. The synthetic promoter of embodiment 36, wherein said promoter comprises the sequence set forth in SEQ ID NO: 1.
38. An expression construct comprising a promoter that drives expression in a plant operably linked to a transcription factor (TF), wherein said nucleotide sequence is selected from sequences having at least 95% identity to the sequences set forth in SEQ ID NOs: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237 and SEQ ID NOS: 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, and 473.
39. A plant transformed with the expression construct of embodiment 38.
40. Transformed seed of the plant of embodiment 39.
41. The plant of embodiment 39, wherein said plant is a monocotyledonous plant.
42. The plant of embodiment 39, wherein said plant is a dicotyledonous plant.
43. The expression construct of embodiment 38, further comprising at least one nucleotide sequence of interest.
44. An expression construct comprising a synthetic promoter operable in a plant cell comprising at least one of the cis-regulatory elements set forth in SEQ IDs No. 475-536 and 543 operably linked to at least one core promoter element operable in a plant, wherein said synthetic promoter is operably linked to a nucleotide sequence.
45. The expression construct of embodiment 44, wherein said nucleotide sequence is a coding sequence.
46. A plant transformed with the expression construct of embodiment 44.
47. Transformed seed of the plant of embodiment 46.
48. The plant of embodiment 46, wherein said plant is a monocotyledonous plant.
49. The plant of embodiment 46, wherein said plant is a dicotyledonous plant.

### FIGURE LEGEND

FIG. 1: Candidate cis-regulatory elements for mesophyll and bundle sheath-specific expression. Putative cis-regulatory elements, “RGCGR” and “WAAAG”, were discovered by ELEMENT and CoGe. The alignment is generated based on sequences from sorghum (Sb03g029170 (SEQ ID NO: 544) and Sb01g040720 (SEQ ID NO: 550)), maize (GRMZM2G121878 (SEQ ID NO: 545) and (GRMZM2G001696 (SEQ ID NO: 549)), rice (Os01g45274 (SEQ ID NO: 547) and LOC.Os03g15050 (SEQ ID NO: 552)) and S. italica (Si003882m (SEQ ID NO: 546) and (Si034404 m.g (SEQ ID NO: 551)) by Multialin (Corpet 1988 Nucleic Acids Res 16: 10881-10890). Boxes highlight the putative elements; box 3 indicates a putative element that is found only in sorghum. The consensus sequence for the alignment in the top panel is set forth in SEQ ID NO: 548, and the consensus sequence for the alignment in the bottom panel is set forth in SEQ ID NO: 553).

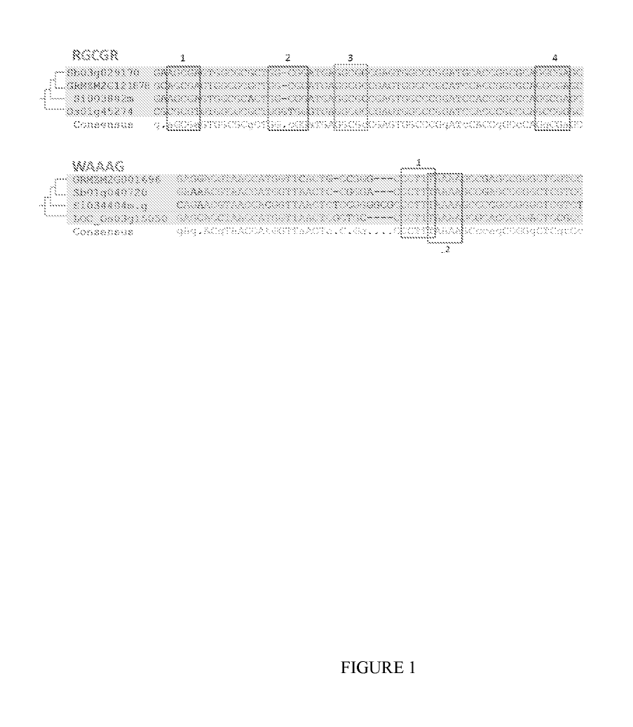

### DETAILED DESCRIPTION OF THE INVENTION

Compositions and methods for the manipulation of photosynthesis through altering the expression of transcription factors (TFs) that regulate genes encoding proteins involved in photosynthesis is provided. The present invention describes methods for identifying a number of transcription factors for the regulation of photosynthetic processes. Without being bound by theory, by altering the expression level and/or profile of one or more of these transcription factors in a plant of interest, photosynthetic metabolism is optimized. Such optimization of photosynthesis provides for increased plant growth and elevated yield in crop plants. The invention provides novel TFs that can be used to transform a plant of interest and can be used in plant breeding programs aimed at developing higher-yielding crops. Recombinant nucleotide sequences encoding the transcription factors are provided. Such methods and elements are disclosed in Wang et al., 2014, Nat. Biotechnol. 32: 1158-1165, which is herein incorporated by reference in its entirety.

A “recombinant polynucleotide” comprises a combination of two or more chemically linked nucleic acid segments which are not found directly joined in nature. By “directly joined” is intended the two nucleic acid segments are immediately adjacent and joined to one another by a chemical linkage. In specific embodiments, the recombinant polynucleotide comprises a polynucleotide of interest or active variant or fragment thereof such that an additional chemically linked nucleic acid segment is located either 5′, 3′ or internal to the polynucleotide of interest. Alternatively, the chemically-linked nucleic acid segment of the recombinant polynucleotide can be formed by deletion of a sequence. The additional chemically linked nucleic acid segment or the sequence deleted to join the linked nucleic acid segments can be of any length, including for example, 1, 2, 3, 4, 5, 6, 7, 8, 9, 10, 15, 20 or greater nucleotides. Various methods for making such recombinant polynucleotides are disclosed herein, including, for example, by chemical synthesis or by the manipulation of isolated segments of polynucleotides by genetic engineering techniques. In specific embodiments, the recombinant polynucleotide can comprise a recombinant DNA sequence or a recombinant RNA sequence. A “fragment of a recombinant polynucleotide” comprises at least one of a combination of two or more chemically linked amino acid segments which are not found directly joined in nature.

A “recombinant polynucleotide construct” comprises two or more operably linked nucleic acid segments which are not found operably linked in nature. Non-limiting examples of recombinant polynucleotide constructs include a polynucleotide of interest or active variant or fragment thereof operably linked to heterologous sequences which aid in the expression, autologous replication, and/or genomic insertion of the sequence of interest. Such heterologous and operably linked sequences include, for example, promoters, termination sequences, enhancers, etc, or any component of an expression cassette; a plasmid, cosmid, virus, autonomously replicating sequence, phage, or linear or circular single-stranded or double-stranded DNA or RNA nucleotide sequence; and/or sequences that encode heterologous polypeptides.

A “recombinant polypeptide” comprises a combination of two or more chemically linked amino acid segments which are not found directly joined in nature. In specific embodiments, the recombinant polypeptide comprises an additional chemically linked amino acid segment that is located either at the N-terminal, C-terminal or internal to the recombinant polypeptide. Alternatively, the chemically-linked amino acid segment of the recombinant polypeptide can be formed by deletion of at least one amino acid. The additional chemically linked amino acid segment or the deleted chemically linked amino acid segment can be of any length, including for example, 1, 2, 3, 4, 5, 6, 7, 8, 9, 10, 15, 20 or amino acids.

By “altering” or “modulating” the expression level of a TF is intended that the expression is upregulated or downregulated. It is recognized that in some instances, plant growth and yield are increased by increasing the expression levels of one or more of the TFs of the invention, i.e. upregulating expression. Likewise, in some instances, plant growth and yield may be increased by decreasing the expression levels of one or more of the TFs of the invention, i.e. downregulating expression. Thus, the invention encompasses the upregulation or downregulation of one or more of the TFs of the invention. Further, the methods include the upregulation of at least one TF and the downregulation of at least one TF in a plant of interest. By modulating the concentration and/or activity of at least one of the TFs of the invention in a transgenic plant is intended that the concentration and/or activity is increased or decreased by at least 1%, 5%, 10%, 20%, 30%, 40%, 50%, 60%, 70%, 80%, or 90% relative to a native control plant, plant part, or cell which did not have the sequence of the invention introduced. It is recognized that the expression levels of the TFs can be controlled by the choice of promoter or the use of enhancers. For example, if a 30% increase is desired, a promoter will be selected to provide the appropriate expression level. The expression level of the TF may be measured directly, for example, by assaying for the level of the TF in the plant. “Transcription factor activity” refers to the ability of a transcription factor to bind to specific DNA sequences, thereby controlling the rate of transcription of genetic information from DNA to messenger RNA.

In order to successfully manipulate the expression level and/or expression profile of candidate TFs, genetic tools, including promoters and enhancer elements, may be used. The present invention describes a number of novel enhancer elements and cis-regulatory elements that were identified through bioinformatic analyses of transcriptomic data. At least one of the enhancer elements may be used to increase the expression of a downstream gene of interest. Alternatively, at least one of the enhancer elements may be combined with a promoter such as a minimal promoter element to create a novel promoter with the desired expression profile.

In many C4 plants, the bundle sheath and mesophyll cells perform very different functions. In these C4 plants, it may be desirable to express a gene or genes of interest in a cell-specific or cell-preferred manner, so that a gene product accumulates primarily in mesophyll or bundle sheath cells. This may be accomplished by transforming a plant of interest with one or more of the cis-regulatory elements described herein. Furthermore, these cis-regulatory elements may be used in C3, C4, or CAM plants to enhance the expression of a gene or genes of interest, whether in a cell-specific or non-cell-specific manner. Still further, these cis-regulatory elements may be used in the design of novel synthetic promoters for the expression of genes of interest. Further, these cis-regulatory elements may be used to alter the expression of a native gene within a plant genome through, e.g., genome-editing approaches.

The compositions of the invention are used to alter expression of genes of interest in a plant, particularly genes involved in photosynthesis. Therefore, the expression of a TF may be modulated as compared to a control plant. A “subject plant or plant cell” is one in which genetic alteration, such as transformation, has been effected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell. Thus, the expression levels are higher or lower than those in the control plant depending on the methods of the invention.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

While the invention is described in terms of transformed plants, it is recognized that transformed organisms of the invention also include plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The enhancers or cis-regulatory elements of the invention can be used to enhance expression of any gene of interest. In one embodiment, the elements can be used with promoters or promoter elements to modulate expression in a plant of interest. Eukaryotic promoters are complex and are comprised of components that include a TATA box consensus sequence at about 35 base pairs 5′ relative to the transcription start site or cap site which is defined as +1. The TATA motif is the site where the TATA-binding-protein (TBP) as part of a complex of several polypeptides (TFIID complex) binds and productively interacts (directly or indirectly) with factors bound to other sequence elements of the promoter. This TFIID complex in turn recruits the RNA polymerase II complex to be positioned for the start of transcription generally 25 to 30 base pairs downstream of the TATA element and promotes elongation thus producing RNA molecules. The sequences around the start of transcription (designated INR) of some polI genes seem to provide an alternate binding site for factors that also recruit members of the TFIID complex and thus “activate” transcription. These INR sequences are particularly relevant in promoters that lack functional TATA elements providing the core promoter binding sites for eventual transcription. It has been proposed that promoters containing both a functional TATA and INR motif are the most efficient in transcriptional activity. (Zenzie-Gregory et al. (1992) J. Biol. Chem. 267:2823-2830). See, also, U.S. Pat. No. 6,072,050, herein incorporated by reference. A “core promoter” or “core promoter element” refers to a minimal region of a regulatory polynucleotide required to properly initiate transcription. A core promoter typically contains the transcription start site (TSS), a binding site for RNA polymerase, and general transcription factor binding sites. Core promoters can include promoters produced through the manipulation of known core promoters to produce artificial, chimeric, or hybrid promoters, and can be used in combination with other regulatory elements, such as cis-elements, enhancers, or introns, for example, by adding a heterologous regulatory element to an active core promoter with its own partial or complete regulatory elements.

The invention encompasses isolated or substantially purified transcription factor or enhancer polynucleotide or amino acid compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived.

Fragments and variants of the disclosed polynucleotides and amino acid sequences encoded thereby are also encompassed by the present invention. By “fragment” is intended a portion of the polynucleotide or a portion of the amino acid sequence. “Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. Generally, variants of a particular polynucleotide of the invention will have at least about 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. Biologically active promoter polynucleotides can have at least about 75%, 80%, 85%, 90%, 91%, 92%, 93%, 94%, 95%, 96%, 97%, 98%, 99% or more sequence identity to the native promoter sequence and retain the ability to initiate transcription (i.e., promoter activity).

“Variant” amino acid or protein is intended to mean an amino acid or protein derived from the native amino acid or protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present invention are biologically active, that is they continue to possess the desired biological activity of the native TF or enhancer. Biologically active variants of a native TF or enhancer sequence of the invention will have at least about 80%, 85%, 90%, 91%, 92%, 93%, 94%, 95%, 96%, 97%, 98%, 99% or more sequence identity to the amino acid sequence for the native sequence as determined by sequence alignment programs and parameters described herein. A biologically active variant of a protein of the invention may differ from that protein by as few as 1-15 amino acid residues, as few as 1-10, such as 6-10, as few as 5, as few as 4, 3, 2, or even 1 amino acid residue.

As indicated, the TFs of the invention can be upregulated or downregulated in a plant of interest. It may be desirable to upregulate at least one TF while simultaneously downregulating at least one different TF. Methods for increasing the expression or upregulating a TF are known in the art and any can be used in the methods of the invention. In one embodiment, upregulation can be achieved by transforming a plant with an expression cassette comprising a promoter operably linked to at least one TF of the invention. Many techniques for upregulating the expression are well known to one of skill in the art, including, but not limited to, designed transcription factors containing a transcriptional activation domain fused to a zinc finger nuclease (Li et al. (2013) Plant Biotechnol J 11: 671-680); dCas9-based transcription factors (Piatek et al. (2015) Plant Biotechnol J 13: 578-589); designed transcription factors containing one or more transcriptional activation domains fused to a DNA-binding protein (Petolino and Davies (2013) Plant Sci 201-202: 128-136); delivery of a virally-derived vector to a plant (Gleba et al. (2014) Curr Top Microbiol Immunol 375: 155-192); each of which is herein incorporated by reference; and other methods or combinations of the above methods known to those of skill in the art.

Downregulation or reduction of the activity of a TF (also known as gene silencing or gene suppression) is also encompassed by the methods of the invention. Many techniques for gene silencing are well known to one of skill in the art, including, but not limited to, antisense technology (see, e.g., Sheehy et al. (1988) Proc. Natl. Acad. Sci. USA 85:8805-8809; and U.S. Pat. Nos. 5,107,065; 5,453,566; and 5,759,829); cosuppression (e.g., Taylor (1997) Plant Cell 9:1245; Jorgensen (1990) Trends Biotech. 8(12):340-344; Flavell (1994) Proc. Natl. Acad. Sci. USA 91:3490-3496; Finnegan et al. (1994) Bio/Technology 12:883-888; and Neuhuber et al. (1994) Mol. Gen. Genet. 244:230-241); RNA interference (Napoli et al. (1990) Plant Cell 2:279-289; U.S. Pat. No. 5,034,323; Sharp (1999) Genes Dev. 13:139-141; Zamore et al. (2000) Cell 101:25-33; and Montgomery et al. (1998) Proc. Natl. Acad. Sci. USA 95:15502-15507), virus-induced gene silencing (Burton et al. (2000) Plant Cell 12:691-705; and Baulcombe (1999) Curr. Op. Plant Bio. 2:109-113); target-RNA-specific ribozymes (Haseloff et al. (1988) Nature 334: 585-591); hairpin structures (Smith et al. (2000) Nature 407:319-320; WO 99/53050; WO 02/00904; WO 98/53083; Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, U.S. Patent Publication No. 20030175965; Panstruga et al. (2003) Mol. Biol. Rep. 30:135-140; Wesley et al. (2001) Plant J. 27:581-590; Wang and Waterhouse (2001) Curr. Opin. Plant Biol. 5:146-150; U.S. Patent Publication No. 20030180945; and, WO 02/00904, all of which are herein incorporated by reference); ribozymes (Steinecke et al. (1992) EMBO J. 11:1525; and Perriman et al. (1993) Antisense Res. Dev. 3:253); oligonucleotide-mediated targeted modification (e.g., WO 03/076574 and WO 99/25853); Zn-finger targeted molecules (e.g., WO 01/52620; WO 03/048345; and WO 00/42219); transposon tagging (Maes et al. (1999) Trends Plant Sci. 4:90-96; Dharmapuri and Sonti (1999) FEMS Microbiol. Lett. 179:53-59; Meissner et al. (2000) Plant J. 22:265-274; Phogat et al. (2000) J. Biosci. 25:57-63; Walbot (2000) Curr. Opin. Plant Biol. 2:103-107; Gai et al. (2000) Nucleic Acids Res. 28:94-96; Fitzmaurice et al. (1999) Genetics 153:1919-1928; Bensen et al. (1995) Plant Cell 7:75-84; Mena et al. (1996) Science 274:1537-1540; and U.S. Pat. No. 5,962,764); dCas9-based transcription factors (Piatek et al. (2015) Plant Biotechnol J 13: 578-589); each of which is herein incorporated by reference; and other methods or combinations of the above methods known to those of skill in the art.

It is recognized that with the polynucleotides of the invention, antisense constructions, complementary to at least a portion of the messenger RNA (mRNA) for the TF sequences can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, optimally 80%, more optimally 85% or greater sequence identity to the corresponding sequences to be silenced may be used. Furthermore, portions of the antisense nucleotides may be used to disrupt the expression of the target gene encoding a TF.

The polynucleotides of the present invention may also be used in the sense orientation to suppress the expression of endogenous genes in plants. The methods generally involve transforming plants with a DNA construct comprising a promoter that drives expression in a plant operably linked to at least a portion of a polynucleotide that corresponds to the transcript of the endogenous gene. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See U.S. Pat. Nos. 5,283,184 and 5,034,323; herein incorporated by reference. Such methods may be used to reduce the expression of at least one TF.

The polynucleotides of the invention can be used to isolate corresponding sequences from other plants. In this manner, methods such as PCR, hybridization, and the like can be used to identify such sequences based on their sequence homology to the sequences set forth herein. Sequences isolated based on their sequence identity to the entire sequences set forth herein or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 75%, 80%, 85%, 90%, 91%, 92%, 93%, 94%, 95%, 96%, 97%, 98%, 99%, or greater sequence identity. Functions of orthologs are often highly conserved among species. Thus, isolated polynucleotides that transcription activation or enhancer activities and which hybridize under stringent conditions to the sequences disclosed herein, or to variants or fragments thereof, are encompassed by the present invention.

Variant sequences can be isolated by PCR as well as hybridization. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

In hybridization techniques, all or part of a known polynucleotide is used as a probe that selectively hybridizes to other corresponding polynucleotides present in a population of cloned genomic DNA fragments from a chosen organism. Methods for hybridization as well as hybridization conditions are generally known in the art and are disclosed in Sambrook et al. (1989) Molecular Cloning: A Laboratory Manual (2d ed., Cold Spring Harbor Laboratory Press, Plainview, N.Y.).

Variant sequences may also be identified by analysis of existing databases of sequenced genomes. In this manner, corresponding TF or enhancer sequences can be identified and used in the methods of the invention.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al. (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See, the world wide web at ncbi.nlm.nih.gov. Alignment may also be performed manually by inspection.

The polynucleotides of the invention can be provided in expression cassettes for expression in a plant of interest. The cassette will include 5′ and 3′ regulatory sequences operably linked to a TF polynucleotide of the invention. “Operably linked” is intended to mean a functional linkage between two or more elements. The cassette may additionally contain at least one additional gene to be co-transformed into the organism. Alternatively, the additional gene(s) can be provided on multiple expression cassettes. Such an expression cassette is provided with a plurality of restriction sites and/or recombination sites for insertion of the TF polynucleotide to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a TF polynucleotide of the invention, and a transcriptional and translational termination region (i.e., termination region) functional in plants.

A number of promoters may be used in the practice of the invention. Constitutive promoters include the CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like, all of which are herein incorporated by reference.

Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Leaf-preferred promoters are also known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

It is recognized that a specific, non-constitutive expression profile may provide an improved plant phenotype relative to constitutive expression of a gene or genes of interest. For instance, many plant genes are regulated by light conditions, the application of particular stresses, the circadian cycle, or the stage of a plant's development. These expression profiles may be highly important for the function of the gene or gene product in planta. One strategy that may be used to provide a desired expression profile is the use of synthetic promoters containing cis-regulatory elements that drive the desired expression levels at the desired time and place in the plant. A number of researchers have identified cis-regulatory elements that can be used to alter gene expression in planta (Vandepoele et al. (2009) Plant Physiol 150: 535-546; Rushton et al. (2002) Plant Cell 14: 749-762). The use of cis-regulatory elements to alter promoter expression profiles has also been reviewed (Venter (2007) Trends Plant Sci. 12: 118-124). The rapid development of new technologies for transcriptomic studies and of new methods to analyze such datasets has enabled the discovery of new cis-regulatory elements. It is well understood that microarray datasets used previously did not have the same resolution as transcriptomic data generated using RNA-Seq. The use of these newer technologies to generate transcriptomic data and the development of new software algorithms for the analysis of transcriptomic data has enabled the discovery of novel cis-regulatory elements including those described herein.

Plant terminators are known in the art and include those available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

As indicated, the TF can be used in expression cassettes to transform plants of interest. Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602 5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. No. 4,945,050; U.S. Pat. No. 5,879,918; U.S. Pat. Nos. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736 740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference. “Stable transformation” or “stable insertion” is intended to mean that the nucleotide construct introduced into a plant integrates into the genome of the plant and is capable of being inherited by the progeny thereof.

The cells that have been transformed may be grown into plants in accordance with conventional ways. See, for example, McCormick et al. (1986) Plant Cell Reports 5:81-84. In this manner, the present invention provides transformed seed (also referred to as “transgenic seed”) having a polynucleotide of the invention, for example, an expression cassette of the invention, stably incorporated into their genome.

The present invention may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatas), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

The following examples are offered by way of illustration and not by way of limitation. All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

### EXPERIMENTAL

### Example 1

### Discovery of 118 Transcription Factors Proposed to Regulate C4 Photosynthesis

Rice and maize leaves were segmented along a developmental gradient, and RNA was extracted from each leaf segment. This RNA was sequenced using RNA-Seq to produce transcriptomic data along the developing leaf gradient. This transcriptomic data was organized into thirty different clusters according to the expression profile for each gene. The expression profiles of the 2,517 maize transcription factors annotated in the Grassius database (on the world-wide web at www.grassius.org) were then analyzed.

The following criteria were applied to discover the transcription factors that are likely to be involved in regulating C4 photosynthesis:

1. The TFs are expressed above background noise in the leaf

2. There is a one to one correspondence of orthologous genes in rice (based on synteny and sequence similarity)

3. There is a consistent profile of differential gene expression in bundle sheath vs. mesophyll cells from two independent cell-type specific datasets (Li et al. (2010) Nat Genet 42: 1060-1069; Chang et al. (2012) Plant Physiol 160: 165-177)

4. The maize TFs and their rice orthologs are mapped to different gene clusters

By applying these four criteria to the developmental transcriptomic data, the list of 118 transcription factors contained in Table 1 was obtained. Without being constrained by theory, it is likely that the differential expression profiles of these transcription factors in rice and maize indicates that these transcription factors may regulate aspects of C4 photosynthesis in maize. The sequences in Table 1 are included in the sequence listing submitted herewith.

TABLE 1
Transcription Factors proposed to regulate aspects of C4 photosynthesis

| Maize gene ID | TF name | Rice ortholog name |
|---|---|---|
| GRMZM2G061906 | ZmbHLH118 | LOC_Os05g38140 |
| GRMZM2G166946 | ZmTCP30 | LOC_Os01g55750 |
| GRMZM2G119999 | ZmHB127 | LOC_Os10g23090 |
| GRMZM2G114137 | ZmCOL8 | LOC_Os03g50310 |
| GRMZM2G177229 | ZmC3H40 | LOC_Os09g31482 |
| GRMZM2G114850 | ZmNAC108 | LOC_Os12g41680 |
| GRMZM2G143204 | ZmWRKY30 | LOC_Os03g55080 |
| GRMZM2G363052 | ZmEREB94 | LOC_Os09g13940 |
| GRMZM2G087804 | ZmGLK13 | LOC_Os01g13740 |
| GRMZM2G024976 | ZmARID6 | LOC_Os02g27060 |
| GRMZM2G701218 | ZmGLK43 | LOC_Os05g40960 |
| GRMZM2G372297 | ZmOrphan69 | LOC_Os01g37130 |
| GRMZM2G130149 | ZmMYB56 | LOC_Os12g37970 |
| GRMZM2G474326 | ZmEREB134 | LOC_Os01g54890 |
| GRMZM2G083347 | ZmNAC67 | LOC_Os12g29330 |
| GRMZM2G380377 | ZmEREB56 | LOC_Os06g03670 |
| GRMZM2G061487 | ZmEREB204 | LOC_Os08g31580 |
| GRMZM2G301089 | ZmbHLH103 | LOC_Os03g53020 |
| GRMZM2G055158 | ZmMYB142 | LOC_Os02g49986 |
| GRMZM2G056120 | ZmARF11 | LOC_Os01g54990 |
| GRMZM2G177220 | ZmARR6 | LOC_Os01g67770 |
| GRMZM2G042895 | ZmbHLH116 | LOC_Os04g23550 |
| GRMZM2G158162 | ZmABI32 | LOC_Os07g48200 |
| GRMZM2G142718 | ZmDOF41 | LOC_Os04g58190 |
| GRMZM2G166721 | ZmNAC16 | LOC_Os01g09550 |
| GRMZM2G118453 | ZmHSF26 | LOC_Os01g54550 |
| GRMZM2G416184 | ZmOrphan192 | LOC_Os09g25330 |
| GRMZM2G149756 | ZmEREB174 | LOC_Os01g64790 |
| GRMZM2G109354 | ZmSBP11 | LOC_Os05g33810 |
| GRMZM2G177386 | ZmOrphan249 | LOC_Os05g46040 |
| GRMZM2G406674 | ZmOrphan33 | LOC_Os05g45930 |
| GRMZM2G377904 | ZmOrphan326 | LOC_Os03g55320 |
| GRMZM2G030762 | ZmbHLH55 | LOC_Os01g68700 |
| GRMZM2G159937 | ZmbHLH57 | LOC_Os01g50940 |
| GRMZM2G154641 | ZmHB48 | LOC_Os01g62920 |
| GRMZM2G160005 | ZmARF27 | LOC_Os06g09660 |
| GRMZM2G143402 | ZmZIM34 | LOC_Os04g55920 |
| GRMZM2G125777 | ZmNAC12 | LOC_Os08g44820 |
| GRMZM2G112629 | ZmbHLH76 | LOC_Os01g01870 |
| GRMZM2G469551 | ZmHB69 | LOC_Os10g33960 |
| GRMZM2G116658 | ZmHB14 | LOC_Os09g35760 |
| GRMZM2G479110 | ZmARR8 | LOC_Os02g55320 |
| GRMZM2G103783 | ZmMYBR82 | LOC_Os09g31454 |
| GRMZM2G008356 | ZmABI22 | LOC_Os07g37610 |
| GRMZM2G405368 | ZmCOL1 | LOC_Os06g16370 |
| GRMZM2G106548 | ZmGRAS54 | LOC_Os07g38030 |
| GRMZM2G149958 | ZmMYB133 | LOC_Os04g58020 |
| GRMZM2G041818 | ZmGBP3 | LOC_Os03g50110 |
| GRMZM2G437460 | ZmARF12 | LOC_Os01g48060 |
| GRMZM2G128206 | ZmOrphan269 | LOC_Os03g15940 |
| GRMZM2G319187 | ZmOrphan151 | LOC_Os01g72330 |
| GRMZM2G025812 | ZmbZIP112 | LOC_Os06g41770 |
| GRMZM2G040115 | ZmOrphan159 | LOC_Os04g57800 |
| GRMZM2G120151 | ZmTCP23 | LOC_Os05g43760 |
| GRMZM2G155954 | ZmOrphan53 | LOC_Os01g61720 |
| GRMZM2G113950 | ZmNAC26 | LOC_Os02g57650 |
| GRMZM2G180979 | ZmC3H17 | LOC_Os03g49170 |
| GRMZM2G135381 | ZmGATA32 | LOC_Os05g44400 |
| GRMZM2G068973 | ZmNAC23 | LOC_Os01g60020 |
| GRMZM2G159996 | ZmOrphan268 | LOC_Os08g42440 |
| GRMZM2G018487 | ZmWRKY107 | LOC_Os03g53050 |
| GRMZM2G026833 | ZmGLK57 | LOC_Os06g24070 |
| GRMZM2G095727 | ZmOrphan208 | LOC_Os03g17570 |
| GRMZM2G010929 | ZmHB26 | LOC_Os01g05080 |
| GRMZM2G108228 | ZmTUB4 | LOC_Os04g59130 |
| GRMZM2G169820 | ZmARF1 | LOC_Os08g40900 |
| GRMZM2G098884 | ZmMYBR83 | LOC_Os06g19980 |
| GRMZM2G110582 | ZmMADS71 | LOC_Os07g27460 |
| GRMZM2G117961 | ZmMADS45 | LOC_Os08g02070 |
| GRMZM2G069713 | ZmOrphan70 | LOC_Os05g29030 |
| GRMZM2G038066 | ZmALF3 | LOC_Os07g41740 |
| GRMZM2G126026 | ZmOrphan270 | LOC_Os05g51690 |
| GRMZM2G134073 | ZmNAC9 | LOC_Os01g70110 |
| GRMZM2G006493 | ZmC3H54 | LOC_Os12g18120 |
| GRMZM2G001577 | ZmOrphan109 | LOC_Os10g30880 |
| GRMZM2G055180 | ZmEREB198 | LOC_Os04g46220 |
| GRMZM2G141299 | ZmWRKY103 | LOC_Os01g54600 |
| GRMZM2G171852 | ZmDOF22 | LOC_Os02g45200 |
| GRMZM2G158252 | ZmOrphan248 | LOC_Os10g21810 |
| GRMZM2G073427 | ZmbZIP111 | LOC_Os12g40920 |
| GRMZM2G079470 | ZmGRAS33 | LOC_Os10g40390 |
| GRMZM2G139688 | ZmMYB138 | LOC_Os01g59660 |
| GRMZM2G421899 | ZmARID5 | LOC_Os02g48370 |
| GRMZM2G169000 | ZmOrphan312 | LOC_Os02g46490 |
| GRMZM2G059851 | ZmHSF6 | LOC_Os02g32590 |
| GRMZM2G159161 | ZmOrphan359 | LOC_Os04g54400 |
| GRMZM2G021777 | ZmCOL3 | LOC_Os02g39710 |
| GRMZM2G314882 | ZmbHLH128 | LOC_Os02g02480 |
| GRMZM2G016749 | ZmOrphan148 | LOC_Os04g33080 |
| GRMZM2G052667 | ZmEREB102 | LOC_Os09g26420 |
| GRMZM2G073892 | ZmbZIP38 | LOC_Os02g58670 |
| GRMZM2G449950 | ZmDOF9 | LOC_Os02g49440 |
| GRMZM2G145041 | ZmMYBR96 | LOC_Os06g51260 |
| GRMZM2G112764 | ZmMYBR26 | LOC_Os08g04840 |
| GRMZM2G028039 | ZmGRAS45 | LOC_Os03g09280 |
| GRMZM2G116785 | ZmbHLH5 | LOC_Os06g09370 |
| GRMZM2G103647 | ZmbZIP17 | LOC_Os06g45140 |
| GRMZM2G481163 | ZmTHX4 | LOC_Os10g41460 |
| GRMZM2G089501 | ZmbHLH106 | LOC_Os09g29930 |
| GRMZM2G126808 | ZmHB45 | LOC_Os03g12860 |
| GRMZM2G097726 | ZmPHD3 | LOC_Os07g08880 |
| GRMZM2G092214 | ZmTCP8 | LOC_Os01g69980 |
| GRMZM2G019106 | ZmbZIP71 | LOC_Os02g03580 |
| GRMZM2G113127 | ZmCA5P13 | LOC_Os08g38780 |
| GRMZM2G384339 | ZmHSF22 | LOC_Os03g63750 |
| GRMZM2G110333 | ZmEREB90 | LOC_Os02g54160 |
| GRMZM2G126832 | ZmOrphan168 | LOC_Os09g14540 |
| GRMZM2G397948 | ZmOrphan73 | LOC_Os01g73750 |
| GRMZM2G006416 | ZmOrphan85 | LOC_Os09g38550 |
| GRMZM2G045544 | ZmPHD22 | LOC_Os02g48810 |
| GRMZM2G414844 | ZmZHD6 | LOC_Os05g50310 |
| GRMZM2G138421 | ZmSBP6 | LOC_Os03g61760 |
| GRMZM2G127537 | ZmHB11 | LOC_Os09g27450 |
| GRMZM2G123876 | ZmC3H30 | LOC_Os03g61110 |
| GRMZM2G161544 | ZmOrphan310 | LOC_Os07g45170 |
| GRMZM2G027972 | ZmWRKY87 | LOC_Os08g38990 |
| GRMZM2G124495 | ZmGLK52 | LOC_Os04g56990 |
| GRMZM2G040452 | ZmOrphan350 | LOC_Os02g13100 |

### Example 2

### Filtering of the 118 Transcription Factors to Prioritize Testing

In order to prioritize the TFs described in Example 1 for further testing, additional filtering was performed. The expression levels of each of the maize TFs and the corresponding rice TFs in Table 1 were compared at each of the 15 gradients in a unified developmental model (UDM). A ratio of maize expression to rice expression level was calculated for each TF pair at each gradient. The maximum and minimum ratios were calculated for each TF pair. The ten TFs from the list of 118 TFs in Table 1 were then selected with the greatest maize:rice expression ratio and the ten TFs from the list of 118 TFs in Table 1 with the smallest maize:rice expression ratio were also selected.

The filtering procedures described here resulted in the selection of twenty maize TFs and their corresponding rice orthologs that show widely divergent expression profiles between rice and maize. These TFs are listed in Table 2. Without being constrained by theory, it is expected that these divergent expression profiles are likely to reflect a change in the function of these TFs in vivo. These twenty TF pairs may be prioritized from the list of 118 TF pairs in Table 1 for further testing and characterization.

TABLE 2
Transcription factor pairs that may be prioritized for additional testing and
characterization

| Largest Maize:Rice Ratios | | | Smallest Maize:Rice Ratios | |
|---|---|---|---|---|
| Maize | Rice | — | Maize | Rice |
| GRMZM2G073892 | LOC_Os02g58670 | | GRMZM2G055180 | LOC_Os04g46220 |
| GRMZM2G079470 | LOC_Os10g40390 | | GRMZM2G117961 | LOC_Os08g02070 |
| GRMZM2G103647 | LOC_Os06g45140 | | GRMZM2G141299 | LOC_Os01g54600 |
| GRMZM2G108228 | LOC_Os04g59130 | | GRMZM2G112629 | LOC_Os01g01870 |
| GRMZM2G397948 | LOC_Os01g73750 | | GRMZM2G159937 | LOC_Os01g50940 |
| GRMZM2G119999 | LOC_Os10g23090 | | GRMZM2G301089 | LOC_Os03g53020 |
| GRMZM2G130149 | LOC_Os12g37970 | | GRMZM2G377904 | LOC_Os03g55320 |
| GRMZM2G142718 | LOC_Os04g58190 | | GRMZM2G469551 | LOC_Os10g33960 |
| GRMZM2G159996 | LOC_Os08g42440 | | GRMZM2G701218 | LOC_Os05g40960 |
| GRMZM2G363052 | LOC_Os09g13940 | | GRMZM2G114850 | LOC_Os12g41680 |

Genes encoding the TFs listed in Table 2 are cloned in a binary vector, operably linked with a promoter functional in a plant cell and a terminator sequence. The binary vector is transformed into Agrobacterium tumefaciens cells, and the A. tumefaciens cells harboring said binary vector are contacted with plant tissue suitable for transformation and regeneration. Following contact with the A. tumefaciens cells, the plant cells are placed on a suitable tissue culture medium for regeneration of plants. These plants are cultivated and assayed for the expression levels of the TF(s) of interest, and the growth characteristics of said plants are assayed in order to determine the effects of TF expression in said plants.

### Example 3

### Derivation of Cis-Regulatory Elements Likely to Drive Expression in a Cell-Specific Manner

A key feature in C4 photosynthesis is the partitioning of photosynthetic activities between two adjacent cell types and in maize this occurs largely through transcriptional control. In a dicot system, it has been shown that a cis-element from a C4 plant can be recognized and confers the same cell-specific pattern of expression in a C3 plant. Thus, one mechanism of differential gene expression appears to be exploiting existing cis-elements conserved between C3 and C4 species. To identify novel cis-elements that drive cell-type specific gene expression in C4 photosynthetic differentiation, promoter sequences were compared between maize and rice from photosynthetic clusters, which include most C4 carbon shuttle genes. Sequences within 3 kb upstream from the start codon of all maize and rice genes in these clusters were examined and then searched for occurrences of ELEMENT-defined motifs in all maize genes from the same cluster. The occurrence (counts) was then tested for enrichment of genes that are highly differentially expressed between BS and ME cells.

A putative cis element (RGCGR; R=A/G) was found to be over-represented in ME-enriched genes in cluster 3. Its presence can be detected upstream of coding regions in several ME-specific carbon shuttle genes, including pyruvate orthophosphodikinase (PPDK), PPDK-regulatory protein (PPDK-RP), Phosphoenol pyruvate carboxylase (PEPC) and carbonic anhydrase (CA). To further examine this putative element, we used CoGe (Lyons and Freeling 2008 Plant J 53: 661-673) to extract synteny-based conserved sequences in the promoter regions of maize, Sorghum, Setaria italica, and rice CA genes. The candidate cis element is found only in promoters of C4 grasses (maize, sorghum and S. italica), but is absent in rice. Interestingly, the motif is present multiple times in the promoter regions of photosynthetic genes of C4 grasses, a feature thought to increase the efficacy of cis-elements (Mehrotra et al. (2005) J Genet 84: 183-187).

A conserved motif “WAAAG” (W=T/A) was enriched in BS-specific genes and appears to be the core component of Dof transcription factors (Yanagisawa and Schmidt (1999) Plant J 17: 209-214). The maize PEPCK gene belongs to cluster 1 and may function in a C4 carbon shuttle (Wingler et al. (1999) Plant Physiol 120: 539-546). We identified a highly conserved non-coding region upstream of PEPCK using CoGe that contained “WAAAG” motifs present as a reverse-complement tandem repeat within the sequence, and is conserved in both C4 and C3 grasses. Interestingly, the native rice PEPCK gene is also expressed in a cell type specific manner (only in BS, vascular and epidermal cells; Nomura et al. (2005) Curr Opin Plant Biol 8:361-368. Thus, unlike the “RGCGR” motif, the “WAAAG” motif was likely recruited from ancestral C3 species to drive cell-specific gene expression. Without being constrained by theory, we speculate that the “WAAAG” element functions combinatorially with other motifs in C4 PEPCK genes to drive high levels of BS-cell specific gene expression. In summary, we have developed a novel algorithm for ELEMENT to define candidate cis-regulatory elements that drive both BS- and M-cell specific gene expressions. Additional candidate cis-regulatory elements are shown in Table 3.

TABLE 3
Candidate cis-regulatory elements that may drive cell-specific expression
of a gene of interest

| BS | ME |
|---|---|
| Candidates | Candidates |
| ACCAGTA | AGCGA |
| CGCGGC | CAGCGG |
| GACCAC | CAACG |
| AGAGATA | ACTCGC |
| ACACAT | CGCCA |
| GGCCACA | AATACG |
| AAAAGC | ACTAG |
| ATGTGTC | ACGTA |
| CACACAC | GCCAC |
| CTCACTC | AGCGTA |
| ACGTCG | GCGAC |
| AAAAGTA | CTTACGCC |
| CGAGCA | AACGC |
| ACGTGA | GACGC |
| GTGTGA | CCGCA |
| GTAGTA | CAGCAGC |
| CACACAC | CACTCG |
| CGCGGC | CTTTCA |
| GACCAC | CGATCGA |
| ACACAT | ATTGTCC |
| TAAAGG | GGCGAA |
| ACGGCC | TAGCTAA |
| ACGTC | ACGAATG |
| CGTTA | CGCATA |
| CTCTCC | ATAGTA |
| ACGAC | ATTTGTC |
| ACGTGC | CGCCACG |
| CGGAAAG | CCAATC |
| AACGT | GGCGA |
| CAACG | AGCGG |
| ACGGA | GGCGG |

### Example 4

### Use of a Cis-Regulatory Element to Construct a Synthetic Promoter that May be Used to Drive a Gene of Interest

The RGCGR element described above was used to construct a novel synthetic promoter. This promoter was used to drive the expression of a gene of interest, resulting in significant accumulation of the encoded mRNA and protein. An approximately 60 bp region at roughly 120 bp upstream of the maize CA1 gene (on the world-wide web at genomevolution.org/r/7hwg) was found to be conserved in sorghum (Sorghum bicolor), maize (Zea mays), foxtail millet (Setaria italica), and rice (Oryza sativa). A novel promoter was constructed using the RGCGR sequence element from sorghum. This sequence element was repeated four times and combined with a minimal promoter element derived from the sorghum carbonic anhydrase gene (S. bicolor chromosome 3: 57333341-57333511). The novel promoter sequence (SEQ ID NO: 1) was termed the 4×RGCGR promoter.

The 4×RGCGR promoter was used to drive expression of a codon-optimized version of the maize SBPase gene (SEQ ID NO: 2) in Brachypodium distachyon and in rice (Oryza sativa). A binary vector plasmid was constructed using standard molecular biology protocols in which the 4×RGCGR promoter was placed upstream of the SBPase open reading frame (SEQ ID NO: 2). This plasmid also contained a selectable marker gene for plant transformation in order to allow for the selection of transformed plant cells. This plasmid was transformed into Agrobacterium tumefaciens cells that were in turn used to transform B. distachyon and O. sativa. The transformed plant cells were regenerated using plant tissue culture techniques. DNA was extracted from the regenerated plants and was tested by PCR to ensure the presence of the full SBPase expression cassette including the 4×RGCGR promoter, the SBPase open reading frame, and other necessary genetic elements to ensure the proper transcription and translation of the transgene.

Following the verification of the presence of the SBPase transgene cassette, leaf samples were collected for protein extraction. Total leaf protein was extracted in standard Tris-buffered saline containing Tween-20 (TBST buffer) and was tested by ELISA for the presence of SBPase protein. The primary antibody was generated in rabbit against a recombinant SBPase protein, and was a gift of Paul Hwang (Washington State University). The secondary antibody was goat anti-rabbit antibody (Thermo-Fisher Scientific). ELISA assays clearly showed a statistically significant increase in SBPase content in the transgenic B. distachyon plants transformed with the 4×RGCGR-SBPase cassette.

The average SBPase content of the seven wild-type B. distachyon plants tested was 0.88±0.27% TSP, while the average SBPase content of the seven B. distachyon plants transformed with the 4×RGCGR-SBPase construct was 6.78±1.54% TSP (Student's t-test p=0.0001, paired, two-tailed distribution).

Protein was also extracted from transgenic rice leaves essentially as described above for B. distachyon, and the resulting protein extracts were tested by ELISA using the protocol described above. For the testing of rice protein extracts, the rice flag leaves were divided into ten equal sections from base to tip with segment 1 at the base and segment 10 at the leaf tip. The protein extracts from each leaf section were tested separately. The results clearly showed that the rice leaf from the plant transformed with the 4×RGCGR-SBPase construct contained significantly more SBPase protein than the leaves of wild-type rice plants.

Transcription profiling was performed on these transgenic rice plants. The flag leaf from each plant was collected and divided into five equal segments. The Trizol (Life Technologies) method was used to extract total RNA. cDNA synthesis was performed using anchored oligo d(T) primers and M-MuLV Reverse Transcriptase (New England BioLabs). qRT-PCR using SYBR green (BioRad Laboratories) was performed with primers specific to the transgenic SBPase (SEQ ID NOs: 537 and 538), the native rice SBPase (SEQ ID NOs: 539 and 540), and a rice control gene (UBQ5) (SEQ ID NOs: 541 and 542). These qRT-PCR experiments clearly showed that the transgenic plants accumulated significant amounts of SBPase transcript driven from the 4×RGCGR promoter.

These results clearly demonstrate that the 4×RGCGR promoter can function effectively in both B. distachyon and O. sativa to drive increased expression of an SBPase gene and accumulation of the encoded SBPase protein. It will be well-understood that the 4×RGCGR promoter is not limited to overexpression of an SBPase gene, but may be used to drive the expression of any gene of interest that has been cloned into a binary vector for plant transformation.

The RGCGR element was used to successfully drive overexpression of a gene of interest by combining this cis-regulatory element with a core promoter element derived from the S. bicolor carbonic anhydrase gene. It will be well-understood by one skilled in the art that other core promoter elements may be used from a wide variety of plant promoters derived from a wide variety of plant species. Such core promoter elements have been described in the scientific literature (Kumari and Ware 2013 PLoS One 8: e79011). The RGCGR cis-regulatory element was derived from bioinformatic analyses of rice and maize transcriptomic data, as described above. Similar analyses uncovered the cis-regulatory elements listed in Table 3. It will be well-understood by one skilled in the art that the cis-regulatory elements listed in Table 3 may be combined with a core promoter element in order to generate novel synthetic promoters that may be used to drive expression of a gene of interest in plant cell.

### Example 5

### Use of Cis-Regulatory Elements to Alter the Expression of a Native Plant Gene

The cis-regulatory elements listed in Table 3 are used to alter the expression of a native plant gene of interest through the use of genome-editing technologies. For this work, at least one copy of one or more of the cis-regulatory elements listed in Table 3 is inserted into a plant genome at a pre-determined site through the use of a site-specific meganuclease or other site-specific insertion method. The insertion site is determined so that the cis-regulatory element is inserted immediately upstream of the core promoter element of the native plant promoter. This strategy is well-understood and has been demonstrated previously to insert a transgene at a predefined location in the cotton genome (D'Halluin et al. (2013) Plant Biotechnol J 11: 933-941). It will be obvious to those skilled in the art that other technologies can be used to achieve a similar result of insertion of genetic elements at a predefined genomic locus (e.g., CRISPR-Cas9, TALENs, and other technologies for precise editing of genomes; Feng et al. (2013) Cell Res 23: 1229-1232, Podevin et al. (2013) Trends Biotechnol 31: 375-383, Wei et al. (2013) J Genet Genomics 40: 281-289, Zhang et al. (2013) Plant Physiol DOI:10.1104/pp. 112.205179). The insertion of one or more copies of one or more of the cis-regulatory elements listed in Table 3 using a technology for genome editing will allow one skilled in the art to manipulate the expression of a downstream native plant gene of interest. The resulting expression profile may show higher or lower expression than the native plant gene. Through the use of the appropriate cis-regulatory elements, a cell-specific expression profile, developmentally-regulated expression profile, circadian cycle-regulated profile, tissue-specific expression profile, inducible expression profile, or other non-constitutive expression profile may be obtained.

### Example 6

### Altering the Expression of Transcription Factors in Planta

The transcription factors listed in Table 1 were derived from bioinformatic analyses of rice and maize developmental transcriptomes. These TFs are proposed to regulate aspects of photosynthesis, which in turn has been linked to plant growth and crop yield (Long et al. (2006) Plant Cell Environ 29: 315-330). By altering the expression level and/or expression profile of one or more of the TFs listed in Table 1, plant growth rates and/or crop yields will be improved.

One or more of the TFs listed in Table 1 may be overexpressed in a crop plant of interest by cloning an open reading frame encoding the TF or TFs of interest downstream of a promoter that is functional in a plant cell. The TF expression cassettes may be transformed into a plant species of interest using a variety of methods including Agrobacterium-mediated transformation or biolistic transformation. It will be well-understood by one skilled in the art that additional techniques to insert DNA into a plant genome may also be used to achieve the goal of overexpression of one or more TFs.

Alternatively, one or more of the TFs listed in Table 1 may be down-regulated using RNAi, amiRNA, or other well-understood techniques to down-regulate the expression of a gene of interest. For these experiments, an RNAi or amiRNA construct designed against the coding region for one or more of the TFs listed in Table 1 is designed and placed downstream of a promoter that is functional in a plant cell. The resulting RNAi or amiRNA cassette or cassettes are cloned into a vector suitable for the transformation of a plant cell and are used to transform the plant species of interest. Said transformation may be realized by using a variety of methods including Agrobacterium-mediated transformation or biolistic transformation. It will be well-understood by one skilled in the art that additional techniques to insert DNA into a plant genome may also be used to achieve the goal of downregulation of one or more TFs in planta.

Alteration of the expression of one or more of the TFs listed in Table 1 may be achieved through the use of precise genome-editing technologies. A nucleic acid sequence will be inserted proximal to a native plant sequence encoding the TF of interest through the use of a meganuclease designed against the plant genomic sequence of interest. This strategy is well-understood and has been demonstrated previously to insert a transgene at a predefined location in the cotton genome (D'Halluin et al. (2013) Plant Biotechnol J 11: 933-941). It will be obvious to those skilled in the art that other technologies can be used to achieve a similar result of insertion of genetic elements at a predefined genomic locus (e.g., CRISPR-Cas9, TALENs, and other technologies for precise editing of genomes; Feng et al. (2013) Cell Res 23: 1229-1232, Podevin et al. (2013) Trends Biotechnol 31: 375-383, Wei et al. (2013) J Genet Genomics 40: 281-289, Zhang et al. (2013) Plant Physiol DOI:10.1104/pp. 112.205179). The insertion of said nucleic acid sequences will be used to achieve the desired result of overexpression of one or more of the TFs listed in Table 1. Alternatively, through the use of the appropriate nucleic acid sequences, downregulation of one or more of the TFs listed in Table 1 may be achieved.

Alteration of the expression of one or more of the TFs listed in Table 1 may also be achieved through the use of self-replicating DNA sequences derived from plant viruses rather than through the stable insertion of the gene or genes of interest into the plant nuclear genome. Sequences derived from plant viruses such as the Geminivirus have been used successfully to achieve expression of multiple genes of interest in a plant (Mozes-Koch et al. (2012) Plant Physiol 158: 1883-1892). By inserting a gene or genes encoding one or more of the TFs listed in Table 1 into a self-replicating construct derived from a plant virus, upregulation of said TFs may be achieved in the plant species of interest by transforming the virus-derived construct into the plant cells and selecting for transformed cells. Alternatively, down-regulation of the TF or TFs of interest selected from the group of TFs listed in Table 1 may be achieved by inserting an amiRNA or RNAi construct designed against one or more of the TFs listed in Table 1 into a plant transformation construct derived from a plant virus. The resulting construct may be transformed into the plant cells of a plant species of interest. Selection for transformed cells and regeneration of plants containing the self-replicating constructs will result in the desired alteration in the expression level and/or expression profile of the TF or TFs of interest.

The TFs listed in Table 1 originate from rice (Oryza sativa) and from maize (Zea mays). The use of one or more of the techniques described herein may lead to an altered expression profile for one or more of the TFs listed in Table 1. Without being limited by theory, it is expected that plant lines with an altered expression profile for one or more of these TFs will exhibit improved plant growth and/or improved crop yield. It will be well-understood to one skilled in the art that closely related homologs or orthologs of the TFs listed in Table 1 may be used in the altered expression strategies described in this Example in order to achieve substantially the same result of an altered TF expression profile leading to improved plant growth and/or improved crop yield. Methods for the identification of orthologous genes have been described in the scientific literature and may be used to identify TFs that are orthologous to the TFs listed in Table 1 (Li et al. (2003) Genome Res 13: 2178-2189; Fulton et al. (2002) Plant Cell 14: 1457-1467). Such orthologous genes may be used in strategies including those described herein to achieve the desired up- or down-regulation of a TF or TFs of interest in the plant species of interest.

### Example 7

### Determining TF Binding Sites

The binding sequences of TFs of interest may be determined through a yeast one-hybrid assay approach. In this approach, the TFs listed in Table 1 are cloned into a vector suitable for protein production in a microbial system (e.g., a pET-series vector; Life Technologies). The TFs are produced in a suitable microbe harboring the protein production plasmid and purified. The purified TFs are screened against a synthetic promoter library in yeast one-hybrid assays. This promoter library contains all 8-mer DNA sequences in at least two different contexts. The results of these yeast one-hybrid assays are the binding sites for the TF being tested. Similar strategies have been described in the scientific literature for the determination of TF binding sites based on yeast one-hybrid assay screening (Pruneda-Paz et al. (2009) Science 323: 1481-1485).

Once the binding sequence has been determined for a TF of interest, this sequence may be used to query the genome of a plant species of interest. Locations within the plant genome that contain the binding sequence for the TF of interest would be likely to interact with the TF in planta. Thus, strategies in which the expression of the TF of interest is altered in the plant of interest would be likely to alter the expression of genes located in close proximity to these binding sites. By locating the nearest open reading frames in either direction from the TF binding site within the plant genome, one skilled in the art could reasonably expect that the expression of these open reading frames would themselves be affected by altering the expression of the TF of interest. Once these open reading frames have been identified, the expression of these genes will be altered directly in planta. Upregulation of these genes will be accomplished by transforming the plants of interest with a vector containing the open reading frame operably linked to a promoter that is operable in a plant cell and then regenerating a transformed plant. The resulting plant will be screened by qRT-PCR, Northern blotting, or other suitable assays to determine transcript levels for the gene or genes that are being overexpressed. Alternatively, downregulation of the genes whose expression is likely to be regulated by the TF of interest will be achieved by transforming a plant species of interest with a plant transformation vector containing an amiRNA sequence designed against these genes. Following transformation, plants will be regenerated, and the resulting plants will be screened by qRT-PCR, Northern blotting, or other suitable assays to determine transcript levels for the gene or genes that are being overexpressed. The growth of transformed plants in which the expression of these genes (i.e., the genes whose expression is regulated by the TFs listed in Table 1) has been altered will be monitored through measurements of the plants. Following maturation of the plants, the total biomass of the plants will be weighed and compared against the total biomass of untransformed wild-type plants. Similarly, the seeds of the transformed plants will be collected and weighed and compared against the total seed weight of untransformed wild-type plants. Without being limited by theory, it is expected that the direct manipulation of the expression of genes whose expression is regulated in part by the TFs listed in Table 1 will cause improved growth and/or improved crop yield in plants.

### Example 8

### Exploring the Mechanism of C₄ Photosynthetic Differentiation Through a Unified Comparative Analysis of Maize and Rice Leaf Transcriptomes

In this study, we explore the leaf transcriptomes of maize (C₄) and rice (C₃), to identify novel structural and regulatory components necessary for photosynthesis. By analyzing the metabolic profiles and correlating orthologous gene expression, we have developed a mathematical model to directly compare two similar developmental gradients and performed cluster analysis to define patterns of maize and rice gene expression. Functional enrichment tests coupled with cis-regulatory mining tools identify candidate motifs likely to have been recruited in the evolution of C₄ photosynthesis. Using these highly resolved transcriptional profiles, we propose a model of suberin biosynthesis—structural features associated with NADP-ME subtype C₄ grasses, and define likely transcriptional regulators of the pathway. We also developed several community tools including an expression viewer to enable broad access to these datasets and provide a foundation for understanding and ultimately engineering C₄ traits into C₃ grasses.

### Results—Metabolic Profiles Along the Maize and Rice Leaf Developmental Gradients

Grass leaves are initiated and develop along a basipetal axis that is distinct from eudicots. This feature facilitates developmental comparisons among different grass species and enables sampling of discrete developmental stages at one fixed time point. Previously, we analyzed the transcriptome of the maize leaf at four developmental stages, and investigated the dynamic changes in gene expression in these segments (Li, P. et al. (2010) Nat Genet 42: 1060-7). In this study, we conduct an inter-specific comparative analysis of photosynthetic differentiation in rice and maize integrating transcriptomics and metabolomics datasets.

Plants used in this study were grown under controlled light, temperature and humidity regimes as previously described (Li, P. et al. (2010) Nat Genet 42: 1060-7). Source and sink boundaries were defined using ¹⁴C labeling in both species using a previously detailed method (Li, P. et al. (2010) Nat Genet 42: 1060-7) and corresponded approximately to the position of the 2^{nd} leaf ligule on leaf 3; leaf segments were then collected from this “anchor point” with defined increments (Methods). To calibrate the leaf gradients, primary and secondary metabolites were measured from 15, 1 cm segments for maize and 11, 2 cm segments for rice. Activities of Calvin Benson cycle enzymes (e.g. Rubisco, NADP-GAPDH) rose >10-fold during leaf development with the increase occurring mainly between segments 2-8. This was closely paralleled by an increase of C₄ enzymes (e.g. PEPC, NADP-malate dehydrogenase; R² between Rubisco and PEPC=0.98). Levels of intermediates involved in intercellular metabolite shuttles also rose between segments 2-8 (R² of the correlation between PEPC and DHAP, 3PGA and pyruvate=0.98, 0.96 and 0.90, respectively). As previously seen, malate unexpectedly peaked in the mid segments and declined at the leaf tip. As expected for a C₃ species, mature rice leaves had higher rates of photorespiration, which was evident from measurements of photorespiratory intermediates including as glycine, glutamine and serine in the photosynthetically active regions of the leaf. This contrasted with maize in which glycine, glutamine and serine were highest in the immature sectors, where the photosynthetic machinery had not yet fully developed. The profiles of most other amino acids also showed opposite trends in the two species. The results in rice support the idea that there is a strong interdependency of nitrogen (N) metabolism and photosynthesis in C₃ plants. C₃ photosynthesis is unavoidably accompanied by photorespiration, which involves the conversion of glycine to serine and the rapid release of ammonium that is re-fixed in parallel with de novo assimilation of nitrate and ammonium (Nunes-Nesi, A. et al. (2010) Mol Plant 3: 973-96; Xu, G. et al. (2012) Annu Rev Plant Biol 63: 153-82). The radically different distribution of amino acids in maize suggests that N metabolism is not tightly coupled to photosynthesis in C₄ plants, and points to the non-photosynthetic leaf sectors at the base playing a predominant role in N assimilation.

### A Unified Developmental Model for Comparative Transcriptomics of Maize and Rice Leaves

Using the same tissues samples from which the metabolite profiles were generated, we performed RNA-seq using a high-throughput library construction protocol (Wang, L. et al. (2011) PloS one 6: e26426) (Methods). An average of 13.8 million 32 bp reads/segment and 207 million reads total were obtained for the maize leaf segments and an average of 22.1 million 32 bp reads/segment and 243 million reads total were obtained for rice. A list of 30,530 maize-rice orthologous genes were generated and used to survey the correlation of gene expression in rice and maize. A heat map profile showing Spearman's rank correlations reveals a similar and continuous transcriptome gradient between maize and rice, yet the different number of segments and the differences identified from metabolic profiles preclude direct comparisons between individual rice and maize leaf segments.

To date, intraspecific analysis of RNA-seq datasets have been limited to post-processing comparisons. That is, network analysis, functional enrichment and transcriptional regulatory components have been performed within species and then datasets compared between species. Here we exploit the uniformity of two highly similar developmental and experimental grass leaf systems to perform an integrated comparative transcriptomics study. To account for the different number of segments sampled along the leaves and variation in developmental progression, we constructed a unified developmental model (UDM) to equate the developmental stages between the two species. Using a core set of 3,559 anchor genes representing high-confidence orthologous gene pairs that have similar profiles of gene expression and likely retain similar functionality in rice and maize (See method for details), we established a common developmental axis onto which segments from both maize and rice could be mapped. This approach preserves the order of segments along leaves and does not force the segments to be equally spaced along the common axis. Given the mapped locations of all the leaf segments, we fitted an expression profile for each maize and rice gene expressed along the common axis. Expression comparisons across species became feasible using the fitted profiles despite the developmental variation and different segment numbers used to profile gene expression. To validate the RNA-seq results and the UDM, we selected 48 maize and rice genes with expression profiles spanning four orders of magnitude and surveyed expression using qRT-PCR. It is evident the RPKM values before and after model fitting are consistent with the qPCR results, with genes expressed at low levels showing more variation than those expressed at high levels. Thus the UDM enables an integrated analysis of maize and rice gene expression data despite 41 million years of evolutionary divergence (on the world-wide web at www.timetree.org). Furthermore, applying the UDM to additional plant and animal systems will be possible when well-calibrated developmental and experimental datasets have been generated.

### Cluster Analysis and the Discovery of Candidate Photosynthesis Cis-Regulatory Elements

To test the efficacy of the UDM we used a modified K-means clustering method (Methods) to examine the expression of genes necessary for photosynthetic differentiation. We generated 30 clusters that capture the major trends along the gradient. The TopGO package (Alexa, A. et al. (2006) Bioinformatics 22: 1600-7) (Methods) identified clusters 1, 3, 4 and 6 as containing genes significantly over-represented with photosynthesis-related GO annotations. Clusters 1, 3 and 4 share similar profiles of gene expression; expression values are low at the base of the leaf peaking at or near the tip of the leaf. In cluster 6, peak expression occurs earlier, near the mid-point of the leaf, representing the source-sink boundary. Genes in cluster 6 include those for tetrapyrrole metabolism, chloroplast targeting and secondary cell wall biosynthesis. (Li, P. et al. (2010) Nat Genet 42: 1060-7; Prioul, J. L. et al. (1980) Plant Physiology 66: 770-4; Miranda, V. et al. (1981) New Phytologist 88: 595-605). Genes in clusters 1, 3 and 4 include those encoding components of the Calvin cycle, photosystems I and II and electron transport. Thus, the expression of genes required for plastid biogenesis precedes the expression of genes required for the implementation of photosynthesis. The UDM indicates that photosynthetic development has proceeded further in rice than maize. This is also evident from the measurements of maize and rice metabolites, as the maize profiles of the starch degradation intermediate maltose (Smith, A. M. et al. (2005) Annu Rev Plant Biol 56: 73-98) and the Calvin-Benson cycle intermediate 3-PGA seem to correspond to the base portions of rice profiles along the leaf gradient. These observations are consistent with the slight enrichment of rice genes in cluster 1 and maize genes in cluster 3, as only correlated regions from the maize and rice leaf were used for clustering.

As the clusters were generated by the UDM, we could exploit the evolutionary distance between maize and rice as a phylogenetic filter to identify conserved cis-elements associated with genes encoding photosynthetic components. A modified ELEMENT algorithm (Mockler, T. C. et al. (2007) Cold Spring Harb Symp Quant Biol 72: 353-63) was developed to incorporate a background correction for multi-species analysis (on the world-wide web at element.mocklerlab.org/). We then searched for motifs associated with photosynthesis in an Arabidopsis cis-element database—AtCOECIS, (Piganeau, G. et al. (2009) J Mol Evol 69: 249-59) as some of the candidates enriched in maize and rice are also conserved in A. thaliana. For instance, from cluster 6, we identified the sequence “ACGTAC” as a motif found upstream of genes associated with photosynthesis (on the world-wide web at bioinformatics.psb.ugent.be/cgi-apps/ATCOECIS/show_motif.htpl?value=GCCACGTN). Similar results were observed in cluster 3, where candidate cis-elements such as “ACGTGTC” (on the world-wide web at bioinformatics.psb.ugent.be/cgi-apps/ATCOECIS/show_motif.htpl?value=CACGTGTC) and “CACGTA” were conserved among maize, rice and A. thaliana. Taken together, the clustering analysis indicates a conservation of putative trans-acting factors that regulate photosynthetic gene expression across angiosperms. However, additional motifs, not conserved between the grasses and A. thaliana may have driven the diversification of photosynthetic development between monocot and dicot lineages.

### Methods

### Plant Growth and RNA-Sequencing Experiment

Maize and rice growth conditions were as previously described (Li, P. et al. (2010) Nat Genet 42: 1060-7; Wang, L. et al. (2011) PloS one 6: e26426). Nine day old third leaves of maize were cut into fifteen 1 cm segments; samples were pooled from an average of seven plants per biological replicate and six biological replicates in total were collected on different dates. 14 day old third leaves of rice were cut into eleven 2 cm segments, samples were pooled from an average of 15 plants per biological replicate and four replicates in total were collected. Total RNA was extracted using TRIzol® (Invitrogen, CA) following manufactory's suggestion. Subsequent RNAseq library construction procedures are detailed in Supplementary File 1. A total of 90 maize and 44 rice leaf libraries were indexed/pooled and sequenced on the Illumina HiSeq 2000® machine, reads were sequenced, deconvoluted and filtered using the manufacture's default pipeline and parameters. The reads were aligned to the maize reference genome B73 AGPv2 using Tophat (Trapnell, C. et al. (2009) Bioinformatics 25: 1105-11). Read counting and calculation of RPKM were described previously, (Wang, L. et al. (2011) PloS one 6: e26426) and later verified with Cuffdiff (Trapnell, C. et al. (2013) Nat Biotechnol 31: 46-53). The variance between replicates is small. The average Pearson correlation between rice replicates is 0.95+−0.07. The average Pearson correlation between maize replicates is 0.97+−0.07. Post-processing of the reads and calculation of RPKM were described previously (Wang, L. et al. (2011) PloS one 6: e26426). Reads were pooled from individual biological replicates to achieve deeper coverage of gene expressed at low levels (Li, P. et al. (2010) Nat Genet 42: 1060-7). The raw reads were uploaded to GEO under accession GSE54274.

### Determination of Maize and Rice Orthologs

Orthologous maize and rice genes were determined first by combining the results from a number of known methods, including BBH-LS (Zhang, M. et al. (2012) BMC Systems Biology 6), Ensembl (Hubbard, T. et al. (2002) Nucleic Acids Res 30: 38-41), MSOAR2 (Shi, G. et al. (2010) BMC Bioinformatics 11: 10), INPARANOID (Ostlund, G. et al. (2010) Nucleic Acids Res 38: D196-203) and ORTHOMCL (Chen, F. et al. (2006) Nucleic Acids Res 34: D363-8). The results from individual methods were assembled into a non-redundant exhaustive list of orthologous pairs in many-to-many relationships that were then filtered to identify one-to-one orthologous gene pairs by choosing the pairs with highest correlation based on non-fitted expression data along the rice and maize leaf gradients.

### Constructing a Unified Maize-Rice Leaf Developmental Model

To define the unified maize-rice leaf developmental gradient and map each leaf section onto this hypothetical coordinate, we developed an iterative computational algorithm as detailed below. Suppose maize leaf segment i (i=1 . . . I) is mapped to the developmental gradient Uᵢ (U₁<U₂< . . . <U_{I}), and rice leaf segment j (j=1 . . . J) mapped to the developmental gradient Vⱼ (V₁<V₂< . . . <V_{J}).

Given the values U=(U₁ . . . U_{I}) and V=(V₁ . . . V_{J}), we model the expected value of the gene expressions (RPKM values) of maize gene g of segment i (X_{gi}) as following:

E(X_{gi})=f(Uᵢ|μ_{g}^{x},α_{g}^{x},β_{g}^{x},γ_{g}^{x})=exp(μ_{g}^{x}+α_{g}^{x}Uᵢ+β_{g}^{x}Uᵢ²+γ_{g}^{x}Uᵢ³).

Similarly, we have the model for rice gene h of segment j (Yₕⱼ):

E(Yₕⱼ)=f(Vⱼ|μₕ^{y},αₕ^{y},βₕ^{y},γₕ^{y})=exp(μₕ^{y}+αₕ^{y}Vⱼ+βₕ^{y}Vⱼ²+γₕ^{y}Vⱼ³).

Note that we use a 3rd-degree polynomial function to model the logarithm of RPKM values. Based on our empirical analysis, this model is adequate and flexible enough to capture most gene expression patterns and at the same time avoid over-fitting. The model parameters μ_{g}^{x} and μₕ^{y} represent the baseline gene expressions at Uᵢ=0 and Vⱼ=0, respectively, for maize gene g and rice gene h. The parameters θ_{g}^{x}=(α_{g}^{x},β_{g}^{x},γ_{g}^{x}) and θₕ^{y}=(αₕ^{y},βₕ^{y},γₕ^{y}) capture the gene expression patterns along the leaf gradients and these parameters are of main interest. The goodness of fit of the model is evaluated by correlation.

Given the values of U and V, we can estimate the expression profiles using the above models. Given a set of genes whose expression profiles are shared between maize and rice, we then can refine the gradients by estimating U and V. Repeating these two steps results in an iterative algorithm. However, we found that some orthologous genes are not suitable for defining the developmental gradients, because they do not share similar expression patterns between the two species. Consequently, we added steps in the algorithm to select the set of “anchor” genes, a subgroup of orthologous genes from the two transcriptomes that share highly similar expression patterns, to unify the developmental gradients. Specifically, we started with two groups of 20,656 maize and 17,634 rice orthologous genes and filtered down to 9,845 one-to-one pairs with highest correlation. We then refined the number of “anchor genes” down to 3,559 using the method described below.

The following iterative algorithm simultaneously select anchor genes, estimate gene expression profiles, and estimate the developmental gradients U and V.

### Algorithm:

1. Initialize. We set Uᵢ=Σₖ₌₁ⁱuₖ and Vⱼ=Σₖ₌₁^{j}vₖ for uₖ>0 and vₖ>0. For the initial step, we set uₖ=1/I and vₖ=1/J for every k.
2. Estimate the shared pattern of ortholog pairs. Denote the set of orthologous gene pairs as O. For any pair (g, h) ε O, we estimate the shared pattern parameters by maximizing the correlation between the observed and predicted gene expressions:

$${θ}_{g}^{*} = {arg max}_{{θ}_{g}} \{ Corr [ {X}_{g} , f ( U \vert {μ}_{g}^{x} , {θ}_{g}^{x} = {θ}_{g} ) ] + Corr [ {Y}_{h} , f ( V \vert {μ}_{h}^{y} , {θ}_{h}^{y} = {θ}_{g} ) ] \}$$

3. Obtain the one-to-one analogous gene pairs. The orthologs were in a many-to-many relationship. We select one to one pairs by two steps. First, we select the rice gene that gives the highest value of Corr[X_{g},P(U|θ_{g}^{x}=θ_{g}*)] when paired with maize gene g as calculated in Step 2. Then, each maize gene is only paired with one rice gene after the first step. Second, among the remaining pairs, we select the maize gene that gives the highest value of Corr[Yₕ,P(V|θₕ^{y}=θ_{g}*)] for each rice gene h i. After the two steps, we get the set of one-to-one ortholog gene pairs, and we denote the set as O*.
4. Select anchor genes. A pair of orthologous genes are selected to be anchor genes if the observed gene expressions for both species have correlation higher than 0.8 with their shared patterns estimated from step 2:

A={(g,h)εO*:Corr[X_{g},\hat{X}_{g}]>0.8&Corr[Yₕ,Ŷₕ]>0.8}

Where \hat{X}_{g} and Ŷ_{g} are the fitted values based on the model estimated from step 2.
5. Refine estimates of gradients using anchor genes. Using the newly defined anchor genes where θ_{g}=θ_{g}*, we re-estimate U and V. As described in step 1, Uᵢ=Σₖ₌₁ⁱuₖ and Vⱼ=Σₖ₌₁^{j}vₖ. We maximize the sum of the correlations between the observed patterns and the fitted ones:

$$\{ {u}_{i} , {v}_{j} \} = {arg max}_{0.9 < \frac{{u}_{i}}{{u}_{i}^{*}} , \frac{{v}_{j}}{{v}_{j}^{*}} < 1.1} \{ {\sum }_{( g , h ) ∈ A} Corr [ {X}_{g} , f ( U \vert {θ}_{g}^{x} = {θ}_{g} ) ] + Corr [ {Y}_{h} , f ( V \vert {θ}_{h}^{y} = {θ}_{g} ) ] \}$$

where uᵢ* and vⱼ* represent the values of uᵢ or vⱼ from previous steps. We search for the refined values of uᵢ and vⱼ within a range of ratios (between 0.9 and 1.1) when compared to their previous values in order to reduce the computation complexity.
6. Iterate. Repeat steps 2-5 until the estimates of U and V become stable. In our analysis, five rounds of iterations were adequate.
   Applying the algorithm described above to the integrative analyses of three or more species is straightforward except that more computation is needed. Additionally, our algorithm is flexible, and it can be adapted to other models of expression profiles and criteria of model fit.

### Co-Clustering the Fitted Maize and Rice Gene Expressions

After establishing the unified gradients, U and V, we fit the expression patterns, f(U|θ_{g}^{x}) and f(V|θₕ^{y}, using gene expressions X_{g} and Yₕ for each gene g of maize and each gene h of rice, respectively.

Before clustering, genes with no clearly defined expression patterns were removed because these genes are of much less interest in the scope of our study due to low expression and/or their noisy nature. Genes whose correlations between observed patterns and fitted ones are above 0.6 are kept in the cluster analysis.

To obtain data vectors to cluster the expression patterns of all selected maize and rice genes, we took N=15 points on the fitted expression profiles for each gene. These points correspond to the same N equally spaced gradients (T₁ . . . T_{N}) with T₁<T₂< . . . <T_{N}, T₁=max{U₁,V₁}, and T_{N}=min{U_{I}, V_{J}}. Hence, only the region shared between maize and rice observed profiles is used for cluster analysis.

A hybrid hierarchical clustering algorithm was used for the cluster analysis. First we performed K-means clustering based on Pearson correlation with K=50. We then merged two clusters with highest correlation based on average linkage at a time. We stopped the merging when none of any two different clusters had an average correlation above 0.9. We obtained K=30 clusters for the final result.

### Functional Enrichment Analysis

Genome annotations was updated using the most recent released data for maize (on the world-wide web at maizesequence.org/index.html) and Rice (on the world-wide web at rice.plantbiology.msu.edu/) as input for the BLAST2GO software (Gotz, S. et al. (2008) Nucleic Acids Res 36: 3420-35). Unique full-length protein sequences were used for BLAST and the resulting GO annotations were converted into the format that is compatible with TopGO package for R¹⁴. We then followed the standard TopGO procedures detailed in its manual (Alexa, A. et al. (2006) Bioinformatics 22: 1600-7) with Fisher's statistical test that generated three tables; they contained the functional enrichment results for all 30 clusters covering three GO classes: Biological Processes, Molecular Functions, and Cellular Components.

Discovering Candidate Cis-Elements with ELEMENT Program

ELEMENT is composed of several modules, each responsible for a single specific task and is invoked separately. First, the “bground” module is used to interrogate background statistics, generally over a set of all promoter sequences in a given species, and is responsible for counting and outputting statistics for each input word or motif over each such promoter sequence. Second, count is used to interrogate foreground statistics, generally over a related subset of all promoters in a given species or group of species, and is responsible for counting and outputting statistics for each input motif, over each input foreground promoter sequence, given the background statistics calculated via “bground”. Third, filter is used to reduce large sets of results to only those that are significant, filter examines each word and respective statistics generated by count, using Benjamini-Hochberg FDR set at 5%, and then outputs only results found to be significant. Fourth, cluster is used to cluster motifs found to be significant by organizing those which are similar. More detailed about ELEMENT can be found on the world-wide web at element.mocklerlab.org.

### Screening Candidate C₄-Related Cis-Elements

We used a combined approach with ELEMENT and enrichment analysis using two-sided Wilcoxon rank-sum statistics to test for potential candidate cis-elements that contribute to cell-type specificity. First we built a list of genes that are enriched either in BS or ME cells in maize leaf tissue based on two previously published datasets (Li, P. et al. (2010) Nat Genet 42: 1060-7; Chang, Y. M. et al. (2012) Plant Physiology). When the differential expression is confirmed in both experiments, we labeled the corresponding gene as cell-type specific. We then counted the occurrences of each cis-elements by ELEMENT from cluster 3 and their reverse-complement sequences in the 3 kb upstream regions of all genes from cluster 3. Wilcoxon rank-sum derived p-values are calculated based on both BS and ME-enriched genes and for all nucleotide patterns. The ones that passed the filter is then visualized using WebLogo (Crooks, G. E. et al. (2004) Genome Res 14: 1188-90)

Validating RNA-Seq Results and the Unified Model Using qRT-PCR

Plant materials for validation were grown independent using same conditions previously described (Li, P. et al. (2010) Nat Genet 42: 1060-7; Wang, L. et al. (2011) PloS one 6: e26426) the soil used for both maize and rice was a mix of 75% Metro 360 and 25% Turface MVP. Three biological replicates of maize and rice were used.

RNA samples from each segment were extracted as previously described (Wang, L. et al. (2011) PloS one 6: e26426). Total RNA was treated by DNase I (Roche, CA) before cDNA synthesis using Transcriptior® First Strand cDNA Synthesis Kit (Roche, CA) and the Anchored Oligo-dT primer. Two cDNA preparations were performed for each sample along with a negative control without reverse transcriptase.

One maize gene and one rice gene were chosen from 14 of the 30 clusters constructed by the model representing various combinations of gene expression levels and cluster sizes. In order to accurately represent the gene-centric RNA seq results, the selected sequences representing all transcript isoforms of the target gene. Primers were designed using Oligo 7®.

Two stable reference genes from maize (GRMZM2G157598) and rice (LOC_Os11g34450) samples were chosen based on the RNA-sequencing results from this and previous publications (Li, P. et al. (2010) Nat Genet 42: 1060-7; Wang, L. et al. (2011) PloS one 6: e26426). Control gene assays gave statistically stable expression across all segments and normalization factors were calculated via geometric averaging of the two control genes' expression levels using BestKeeper® software. Quantification of target gene expressions were calculated relative to the calibrator gene using advanced relative quantification in the LightCycler® 480 SW 1.5 software (Roche, CA). In each segment, the ratio of the expression level of the calibrator gene to the target gene was calculated using the software. The ratios were plotted as expression patterns along the developmental segments. Reactions were run with LightCycler® 480 SYBR Green I Master (Roche, CA) in a Roche LightCycler® 480 II Real-Time PCR machine using following program: 95° C. for 5 minutes, 45 cycles of 10 seconds at 95° C., 10 seconds at 60° C., and 10 seconds at 72° C., followed by 1 cycle of 95° C. for 5 seconds and 65° C. for one minute, samples were left at 95° C. without cooling. Three technical replicates of each sample were included in the qPCR experiment.

### Measurements of Maize and Rice Metabolites

Leaf samples from maize and rice plants used for metabolite measurements were grown at the same conditions as described previously (Li, P. et al. (2010) Nat Genet 42: 1060-7; Wang, L. et al. (2011) PloS one 6: e26426). Sections from 20-30 plants were pooled for each maize sample and 30-40 for each rice sample. Six biological samples were prepared for maize and four for rice. Frozen leaf material at −80° C. was ground to a fine powder using a cryogenic grinding robot prototype (Labman, Newcastle, UK). Sample sub-aliquots for the different analyses were either weighted by the robot or by hand using an analytical balance, and were constantly kept at freezing temperatures.

Secondary metabolite analysis by LC-MS was performed on HPLC system Surveyor (Thermo Finnigan, USA) coupled to Finnigan LTQ-XP system (Thermo Finnigan, USA) as described by Tohge and Fernie. All data were processed using Xcalibur 2.1 software (Thermo Fisher Scientific, Waltham, USA). The obtained data matrix of peak area was normalized using the internal standard (sinigrin, CAS: 3952-98-5). Metabolite identification and annotation were performed using metabolite databases and literature survey of Zea mays (Elliger, C. A. et al. (1980) Phytochemistry 19: 293-297; Snook, M. E. et al. (1995) Journal of Agricultural and Food Chemistry 43: 2740-2745) and monocot species (Tohge, T. et al. (2011) Plant Physiology 157: 1469-1482; Matsuda, F. et al. (2012) Plant Journal 70: 624-636). GC-MS metabolite profiling and carbon starvation experiments were conducted as described in Tohge et al. (Tohge, T. et al. (2011) Plant Physiology 157: 1469-1482). Enzyme activity measurements were conducted as previously described (Gibon, Y. et al. (2004) Plant Cell 16: 3304-3325; Sulpice, R. et al. (2010) Plant Cell 22: 2872-2893) using an established semi-robotized 96 well micro-titer plate platform.

### Example 9

### Expression of Transcription Factors in Planta

Eleven of the transcription factors were chosen for over expression in rice and the model C4 grass Setaria viridis (Table 4). These transcription factors were cloned into expression cassettes in binary vectors capable of maintenance in E. coli and A. tumefaciens. The two expression cassettes include the 2×355 and the ZmRbcS promoters, respectively. Additionally, all eleven transcription factors are cloned under the ZmCA1 promoter. Two TFs (GRMZM2G127537 and GRMZM2G124495) were cloned under the ZmPepC promoter. All binary vectors were transformed into A. tumefaciens strain LBA4404. The A. tumefaciens cells harboring the appropriate binary vectors are used for transformation of S. viridis and of O. sativa cells. Following transformation of plant cells, appropriate tissue culture techniques are used to regenerate fertile plants.

TABLE 4
Binary Vectors Cloned

| Construct | | | |
|---|---|---|---|
| ID | Promoter + 5′UTR | ORF | 3′UTR |
| 130576 | 2X 35S | GRMZM2G010929 | 35S poly A |
| 130577 | 2X 35S | GRMZM2G127537 | 35S poly A |
| 130578 | 2X 35S | GRMZM2G124495 | 35S poly A |
| 130579 | 2X 35S | GRMZM2G416184 | 35S poly A |
| 130678 | ZmRbcS | GRMZM2G141299 | ZmRbcS |
| 130679 | ZmRbcS | GRMZM2G169820 | ZmRbcS |
| 130680 | ZmRbcS | GRMZM2G159161 | ZmRbcS |
| 130681 | ZmRbcS | GRMZM2G019106 | ZmRbcS |
| 130682 | ZmRbcS | GRMZM2G119999 | ZmRbcS |
| 130683 | ZmRbcS | GRMZM2G177229 | ZmRbcS |
| 130684 | ZmRbcS | GRMZM2G061906 | ZmRbcS |
| 130685 | ZmRbcS | GRMZM2G130149 | ZmRbcS |
| 130694 | 2X 35S | GRMZM2G141299 | 35S poly A |
| 130695 | 2X 35S | GRMZM2G169820 | 35S poly A |
| 130696 | 2X 35S | GRMZM2G159161 | 35S poly A |
| 130697 | 2X 35S | GRMZM2G019106 | 35S poly A |
| 130698 | 2X 35S | GRMZM2G119999 | 35S poly A |
| 130699 | 2X 35S | GRMZM2G177229 | 35S poly A |
| 130700 | 2X 35S | GRMZM2G061906 | 35S poly A |
| 130701 | 2X 35S | GRMZM2G130149 | 35S poly A |
| 130785 | ZmRbcS | GRMZM2G010929 | ZmRbcS |
| 130786 | ZmRbcS | GRMZM2G127537 | ZmRbcS |
| 130787 | ZmRbcS | GRMZM2G124495 | ZmRbcS |
| 130788 | ZmRbcS | GRMZM2G416184 | ZmRbcS |
| 130790 | ZmPepC | GRMZM2G127537 | ZmPepC |
| 130791 | ZmPepC | GRMZM2G124495 | ZmPepC |
| 130686 | ZmCA | GRMZM2G141299 | ZmCA |
| 130687 | ZmCA | GRMZM2G169820 | ZmCA |
| 130688 | ZmCA | GRMZM2G159161 | ZmCA |
| 130689 | ZmCA | GRMZM2G019106 | ZmCA |
| 130690 | ZmCA | GRMZM2G119999 | ZmCA |
| 130691 | ZmCA | GRMZM2G177229 | ZmCA |
| 130692 | ZmCA | GRMZM2G061906 | ZmCA |
| 130693 | ZmCA | GRMZM2G130149 | ZmCA |
| 130792 | ZmCA | GRMZM2G416184 | ZmCA |

Tables 5 and 6 show the constructs that were used to transform S. viridis and O. sativa, respectively. Construct 130790 was successfully transformed into Setaria viridis. Gene copy number analysis via quantitative PCR was used to screen for single copy integration events. FAM-ZEN/Iowa Black FQ probe and primers (IDT DNA) were designed for the Setaria viridis PCK gene, previously shown to be single copy (Xu et al. (2013) Plant Mol Biol 83: 77-87). Quantitative PCR was performed using iQ Supermix Master Mix (Biorad Laboratories). Six of the ten events were shown to be single copy integrations.

TABLE 5
Constructs Transformed into Setaria viridis

| 6-Digit | | | |
|---|---|---|---|
| ID | Promoter + 5′UTR | ORF | 3′UTR |
| 130577 | 2X 35S | GRMZM2G127537 | 35S poly A |
| 130579 | 2X 35S | GRMZM2G416184 | 35S poly A |
| 130681 | ZmRbcS | GRMZM2G019106 | ZmRbcS |
| 130682 | ZmRbcS | GRMZM2G119999 | ZmRbcS |
| 130683 | ZmRbcS | GRMZM2G177229 | ZmRbcS |
| 130684 | ZmRbcS | GRMZM2G061906 | ZmRbcS |
| 130685 | ZmRbcS | GRMZM2G130149 | ZmRbcS |
| 130694 | 2X 35S | GRMZM2G141299 | 35S poly A |
| 130698 | 2X 35S | GRMZM2G119999 | 35S poly A |
| 130699 | 2X 35S | GRMZM2G177229 | 35S poly A |
| 130700 | 2X 35S | GRMZM2G061906 | 35S poly A |
| 130701 | 2X 35S | GRMZM2G130149 | 35S poly A |
| 130790 | ZmPepC | GRMZM2G127537 | ZmPepC |
| 130791 | ZmPepC | GRMZM2G124495 | ZmPepC |

TABLE 6
Constructs Transformed into O. sativa

| 6-Digit | | | |
|---|---|---|---|
| ID | Promoter + 5′UTR | ORF | 3′UTR |
| 130681 | ZmRbcS | GRMZM2G019106 | ZmRbcS |
| 130682 | ZmRbcS | GRMZM2G119999 | ZmRbcS |
| 130683 | ZmRbcS | GRMZM2G177229 | ZmRbcS |
| 130684 | ZmRbcS | GRMZM2G061906 | ZmRbcS |
| 130685 | ZmRbcS | GRMZM2G130149 | ZmRbcS |
| 130698 | 2X 35S | GRMZM2G119999 | 35S poly A |
| 130699 | 2X 35S | GRMZM2G177229 | 35S poly A |
| 130700 | 2X 35S | GRMZM2G061906 | 35S poly A |
| 130701 | 2X 35S | GRMZM2G130149 | 35S poly A |

We claim:

1. A method of improving plant growth by altering the expression of at least one nucleotide sequence encoding a transcription factor (TF), wherein said nucleotide sequence is selected from sequences having at least 95% identity to the sequences set forth in SEQ ID NOs: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, and SEQ ID NOS: 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, and 473, wherein said nucleotide sequence retains transcription factor activity.

2. The method of claim 1 wherein said at least one transcription factor is upregulated such that expression of the TF is increased relative to a control plant cell.

3. The method of claim 1 wherein said at least one transcription factor is downregulated such that expression of the TF is decreased relative to a control plant cell.

4. The method of claim 1, wherein said altering is achieved by the stable insertion of at least one expression construct comprising a promoter that drives expression in a plant, operably linked to at least one nucleotide sequence encoding at least one transcription factor.

5. The method of claim 4, wherein said promoter is a constitutive promoter.

6. The method of claim 4, wherein said promoter is a non-constitutive promoter.

7. A synthetic promoter operable in a plant cell comprising at least one of the cis-regulatory elements set forth in SEQ IDs No. 475-536 and 543 operably linked to at least one core promoter element operable in a plant cell.

8. The synthetic promoter of claim 7, wherein said promoter comprises the sequence set forth in SEQ ID NO: 1.

9. A method of expressing at least one sequence of interest in a plant by transforming said plant with a construct comprising the synthetic promoter of claim 7 operably linked to at least one coding sequence of interest.

10. The method of claim 1, wherein the plant of interest is a monocotyledonous plant.

11. The method of claim 1, wherein the plant of interest is a dicotyledonous plant.

12. An expression construct comprising a promoter that drives expression in a plant operably linked to a transcription factor (TF), wherein said nucleotide sequence is selected from sequences having at least 95% identity to the sequences set forth in SEQ ID NOs: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237 and SEQ ID NOS: 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, and 473, wherein said nucleotide sequence retains transcription factor activity.

13. A plant transformed with the expression construct of claim 12.

14. Transformed seed of the plant of claim 13.

15. The plant of claim 13, wherein said plant is a monocotyledonous plant.

16. The plant of claim 13, wherein said plant is a dicotyledonous plant.

17. The expression construct of claim 12, further comprising at least one nucleotide sequence of interest.

18. An expression construct comprising a synthetic promoter operable in a plant cell comprising at least one of the cis-regulatory elements set forth in SEQ IDs No. 475-536 and 543 operably linked to at least one core promoter element operable in a plant, wherein said synthetic promoter is operably linked to a nucleotide sequence.

19. The expression construct of claim 18, wherein said nucleotide sequence is a coding sequence.

20. A plant transformed with the expression construct of claim 18.

21. Transformed seed of the plant of claim 20.

22. The plant of claim 20, wherein said plant is a monocotyledonous plant.

23. The plant of claim 20, wherein said plant is a dicotyledonous plant.
